(19) United States
(12) Patent Application Publication
(10) Pub. No.: US 2020/0188178 A1
(43) Pub. Date: Jun. 18, 2020

(54) DOUBLE-LAYER DRESSING CONTAINING SILK FIBROIN AND A METHOD FOR MAKING THE SAME

(71) Applicants: National Taiwan University of Science and Technology, Taipei City (TW); LIFE STAR INTERNATIONAL LIMITED, New Taipei City (TW)

(72) Inventors: MENG-YI BAI, Taoyuan City (TW); MENG-CHUAN CHEN, New Taipei City (TW); JIA-YING LIN, New Taipei City (TW); WEI-YIN CHEN, Changhua County (TW)

(21) Appl. No.: 16/224,441

(22) Filed: Dec. 18, 2018

(51) Int. Cl. A61F 13/00; A61F 13/84; B32B 5/26; B32B 5/06

(52) U.S. Cl. CPC A61F 13/00012; A61F 13/8405; A61F 13/00029; A61F 13/00987; B32B 2535/00; B32B 5/06; A61F 2013/8438; B32B 2262/08; B32B 5/26

(57) ABSTRACT

Disclosures of the present invention describe a double-layer dressing containing silk fibroin and a method for making the same. The double-layer dressing mainly comprises a silk fibroin layer and a calcium-degradation silk fibroin layer connected to the silk fibroin layer. It is worth emphasizing that, results of animal experiment have proved that this novel double-layer dressing is an outstanding hemostatic wound dressing. Moreover, additional adhesion, resulted from the solidification of tissue fluid, can be effectively prevented from forming between skin wound and wound dressing under the use of this double-layer dressing.

### BACKGROUND OF THE INVENTION

### 1. Field of the Invention

The present invention relates to the technology field of wound dressings, and more particularly to a double-layer dressing containing silk fibroin and a method for making the same.

### 2. Description of the Prior Art

According to a narrow definition, a wound is a type of injury which happens in skin. In a normal case, platelets would immediately achieve a hemostasis by coagulating bleeding of a wound after the occurrence of an injury to skin. However, since some people show a poor hemostasis result in their skin wounds, various hemostatic products are hence developed so as to be further provided for use in enhancing the hemostasis efficiency on skin wounds. There are two methods commonly used to stop bleeding. One way is directly compressing a bleeding blood vessel for the stop of bleeding, and the other way is tightly wrapping a tourniquet around the bleeding arm or leg about 2 to 3 inches above the bleeding site. It needs to particularly note that, the wound must be firstly covered by a clean cloth (for example, a shirt), such that directly compressing a bleeding blood vessel can be achieved by apply continuous pressure with both hands on the clean cloth. Band-Aid, one kind of wound dressing, is well-known an adhesive bandage for use in covering the skin wound and directly compressing the bleeding blood vessel.

Recently, various hemostatic dressings made by different natural materials have been widely developed. There are several natural materials used in the manufacture of the hemostatic dressings are known including Gelatin, fibrin, and chitosan, wherein silk fibroin is one kind of natural fibrin. China patent publication No. CN103497240A has disclosed a method for manufacturing a peptide powder hemostatic product using silk fibroin, comprising following steps:
(1) cutting a silkworm cocoon into a plurality of silk slices, and then putting the silk slices into a neutral NaOH solution of 2% for executing a fabric refining process;
(2) using a sodium carbonate solution of 0.05 wt % and deionized water to repeatedly wash the silk slices during the execution of the fabric refining process, and simultaneously using a picrocarmine solution of 0.5% to test whether the silk slices have been degummed or not;
(3) dissolving the degummed silk fibers in a salt aqueous based on a mixing ratio of 1:20, and then heating the mixture solution by a treatment temperature of 80-180° C. and a treatment pressure of 100-500 kPa, so as to obtain a silk fibroin solution; and
(4) applying a freeze-drying process to the silk fibroin solution, thereby obtaining a peptide powder hemostatic product.

In the step (3), the said salt solution is produced by mixing NaCl, ethanol and water based on a weight ratio of 0.1-1:0.1-1:1. Moreover, engineers skilled in the manufacture of silk fibroin products should know that, the peptide powder hemostatic product obtained from the step (5) is actually a hydrolysis intermediate of the silk fibroin. On the other hand, from the disclosures of the China patent publication No. CN103497240A, it is clearly known that the hemostasis time of the peptide powder hemostatic product is around 87±11 seconds, which is almost equal to that of the commercial powder hemostatic product Arista.

In spite of the fact that the disclosed peptide powder hemostatic product exhibits a good hemostasis efficiency, the peptide powder hemostatic product is still found to show drawbacks in practical use. The drawbacks are summarized as follows.
(1) After the fine-granular peptide powder is applied to a skin wound, tissue adhesion may still occur between the skin wound and the peptide powder, thereby making user feel uncomfortable.
(2) Following on from the previous descriptions, it is extrapolated that the user may certainly further feel uncomfortable after apply continuous pressure on the peptide powder hemostatic product covering the skin wound.

Accordingly, in view of the conventional peptide powder hemostatic product still showing many drawbacks and shortcomings in practical applications, the inventors of the present application have made great efforts to make inventive research thereon and eventually provided a double-layer dressing containing silk fibroin and a method for making the same.

### SUMMARY OF THE INVENTION

The primary objective of the present invention is to provide a double-layer dressing containing silk fibroin and a method for making the same. The double-layer dressing mainly comprises a silk fibroin layer and a calcium-degradation silk fibroin layer connected to the silk fibroin layer. It is worth emphasizing that, results of animal experiment have proved that this novel double-layer dressing is an outstanding hemostatic wound dressing. Moreover, additional adhesion, resulted from the solidification of tissue fluid, can be effectively prevented from forming between skin wound and wound dressing under the use of this double-layer dressing.

In order to achieve the primary objective of the present invention, the inventor of the present invention provides an embodiment for the double-layer dressing containing silk fibroin, comprising:

a silk fibroin layer; and
a calcium-degradation silk fibroin layer, being disposed on the silk fibroin layer.

In the aforesaid embodiment of the double-layer dressing containing silk fibroin, a substrate is used for supporting the silk fibroin layer by the surface thereof.

Moreover, for achieving the primary objective of the present invention, the inventor of the present invention provides an embodiment for the method for making the double-layer dressing containing silk fibroin, comprising following steps:
(1) preparing a raw material of silkworm cocoon, and then producing a silk fibroin product by applying a degumming process to the raw material of silkworm cocoon;
(2) dissolving the silk fibroin product in a salt solution, so as to obtained a silk fibroin solution;
(3) applying a dialysis process to the silk fibroin solution, thereby obtaining a dialysate of silk fibroin;
(4) separating the dialysate of silk fibroin into a first dialysate and a second dialysate;
(5) producing a first supernatant by applying a first centrifuging process to the first dialysate;
(6) applying a degradation process to the second dialysate so as to obtain a protein-degraded solution, and subsequently producing a mixture solution by mixing the protein-degraded solution with a solution of bivalent metal salt;
(7) producing a second supernatant by applying a second centrifuging process to the mixture solution;
(8) applying a freeze-drying process to both the first supernatant and the second supernatant thereby respectively obtaining a silk fibroin layer and a calcium-degradation silk fibroin layer, and then producing a double-layer dressing containing silk fibroin by disposing the calcium-degradation silk fibroin layer on the silk fibroin layer.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a schematic stereo diagram of a first embodiment of a double-layer dressing containing silk fibroin according to the present invention;

FIG. 2 shows a schematic application diagram of the double-layer dressing containing silk fibroin;

FIG. 3A and FIG. 3B show a flow chart of a method for making the double-layer dressing containing silk fibroin according to the present invention;

FIG. 4A and FIG. 4B show diagrams for describing manufacturing processes of the double-layer dressing containing silk fibroin;

FIG. 5 shows a sample of silk fibroin layer being subject to a mechanical testing

FIG. 6 shows a sample of calcium-degradation silk fibroin layer being subject to a mechanical testing;

FIG. 7 shows a plot graph of mmHg versus testing time;

FIG. 8 shows a plot graph of mmHg versus testing time;

FIG. 9 shows a statistical bar graph of various test samples for describing blood absorption ability of the test samples; and

FIG. 10 shows a statistical bar graph of various test samples for describing blood 0absorption ability of the test samples.

### DETAILED DESCRIPTION OF THE PREFERRED EMBODIMENTS

To more clearly describe a double-layer dressing containing silk fibroin according to the present invention, embodiments of the present invention will be described in detail with reference to the attached drawings hereinafter.

### First Embodiment

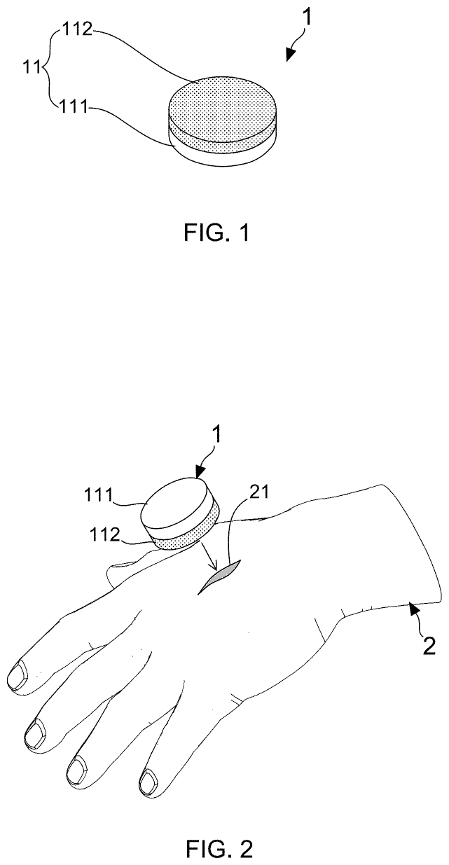

With reference to FIG. 1, which shows a schematic stereo diagram of a first embodiment of a double-layer dressing containing silk fibroin according to the present invention. Moreover, FIG. 2 shows a schematic application diagram of the double-layer dressing containing silk fibroin. As FIG. 1 shows, the first embodiment of the double-layer dressing 1 containing silk fibroin comprises a silk fibroin layer 111 and a calcium-degradation silk fibroin layer 112 disposed on the silk fibroin layer 111. When using this double-layer dressing 1, the double-layer dressing 1 is attached to a skin area 2 by the calcium-degradation silk fibroin layer 112 thereof, so as to make the calcium-degradation silk fibroin layer 112 contact and cover a wound 21 forming on the skin area 2.

Manufacturing Method

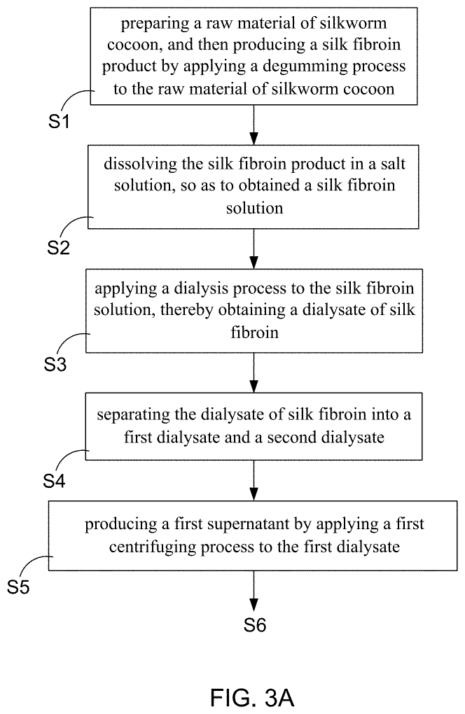

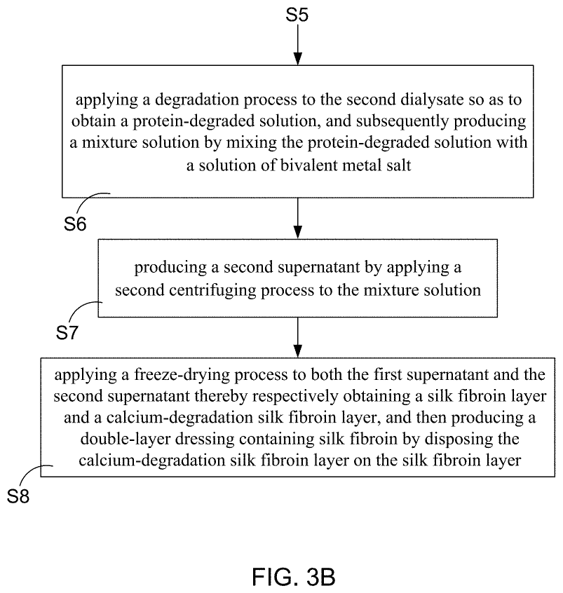

Please refer to FIG. 3A and FIG. 3B, there are shown a flow chart of a method for making the double-layer dressing containing silk fibroin according to the present invention. As FIG. 3A shows, the method flow is firstly proceeded to step S1: preparing a raw material of silkworm cocoon, and then producing a silk fibroin product by applying a degumming process to the raw material of silkworm cocoon. During the execution of step S1, the silkworm cocoon or the slices of the silkworm cocoon are disposed in a Na₂CO₃ solution of 0.05 g/L, so as to be heated by a treatment temperature of 85-100° C. for 2-3 hours. Subsequently, a weak base is added into the solution for adjusting pH value of the solution to 9-11, and then the solution is further applied with an ultrasonic process under 70-80° C. for 90-120 minutes. There is another way for completing the degumming process. Firstly, the silkworm cocoon or the slices of the silkworm cocoon are disposed in sodium dodecyl sulfate (SDS) solution with the concentration of 0.05 g/L, and then the solution is heated by a treatment temperature of 85-100° C. for 1 hour. Subsequently, after being washed by deionized water, the slices of the silkworm cocoon are further putting into Na₂CO₃ solution with concentration of 0.25%, and then the solution is heated by a treatment temperature of 85-100° C. for 1 hour. Consequently, the slices of the silkworm cocoon are removed from the Na₂CO₃ solution, and then washed by deionized water.

Above descriptions are presented for emphasizing that the present invention does not limits the detail executing steps or ways for completing the degumming process of the step S1. As FIG. 3A shows, the method flow is next proceeded to step S2, so as to dissolve the silk fibroin product in a salt solution, thereby obtaining a silk fibroin solution. In the present invention, the salt in the salt solution is a neutral salt, such as a compound of Li and chloride, bromide or iodide, a compound of Sr and chloride, bromide or iodide, or a compound of Ba and chloride. On the other hand, solution comprising calcium and polyols is also adopted for dissolving the silk fibroin, such as a ternary solution produced by mixing CaCl₂), ethanol and water with a mole ratio of 1:2:8. Therefore, it is apparent that the present invention does not limits the detail executing steps or ways for dissolving the silk fibroin.

The method flow subsequently proceeds step S3 and step S4. In the step S3, the silk fibroin solution is applied with a dialysis process in order to obtain a dialysate of silk fibroin. In the step S4, the obtained dialysate of silk fibroin is further separated into a first dialysate and a second dialysate. Next, the method flow is proceeded to step S5 and step S6. In the step S5, there is a first supernatant obtained by applying a first centrifuging process to the first dialysate. Moreover, in the step S6, it applies a degradation process to the second dialysate so as to obtain a protein-degraded solution, and then a mixture solution is produced by mixing the protein-degraded solution with a solution of bivalent metal salt. Furthermore, in step S7, there is a second supernatant obtained by applying a second centrifuging process to the mixture solution.

In the step S6, the degradation process is achieved by adding a protease solution comprising a buffer solution and a protease into the second dialysate, wherein phosphate buffered saline (PBS) and deionized water are commonly used as the buffer solution. Moreover, the protease is selected from the group consisting of protease produced by Bacillus amyloliquefaciens, protease produced by Streptomyces griseus, α-chymotrypsin, chymotrypsin, and carboxylase. In the present invention, the protease used in the step S6 must has a molecular weight in a range from 25000 daltons to 35000 daltons. On the other hand, the salt solution used in the step S6 is a solution of bivalent metal salt comprises a bivalent metal salt and a solution, and having a solution concentration of at least 0.2 mg/mL. For example, the solution is water, and the bivalent metal salt can be zinc chloride, calcium chloride or magnesium chloride. On the other hand, solution comprising calcium and polyols is also adopted for dissolving the silk fibroin, such as a ternary solution produced by mixing CaCl₂), ethanol and water with a mole ratio of 1:2:8. Therefore, it is apparent that the present invention does not limits the detail executing steps or ways for dissolving the silk fibroin.

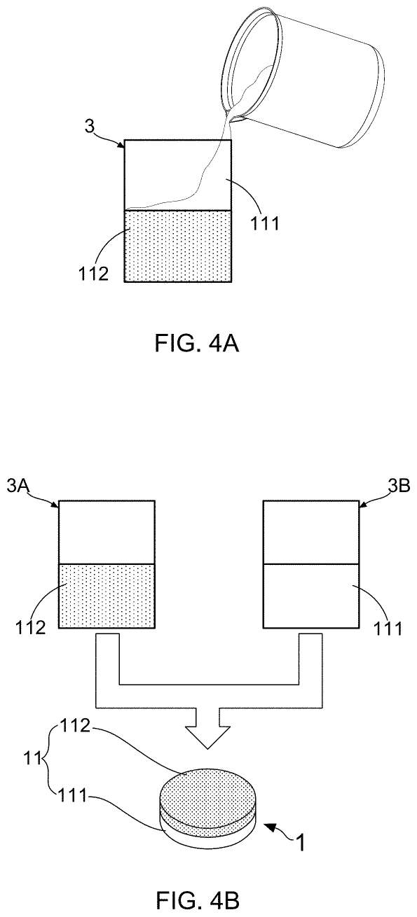

Consequently, the method flow proceeds to step S6 for applying a freeze-drying process to both the first supernatant and the second supernatant thereby respectively obtaining a silk fibroin layer and a calcium-degradation silk fibroin layer, thereby producing a double-layer dressing containing silk fibroin by disposing the calcium-degradation silk fibroin layer on the silk fibroin layer. Please refer to FIG. 1 again, and also simultaneously refer to FIG. 4A and FIG. 4B showing diagrams for describing manufacturing processes of the double-layer dressing containing silk fibroin. Diagram in FIG. 4A depicts that a second supernatant obtained from the step S7 is filled into a mold 3 such as a paper cup, and a calcium-degradation silk fibroin layer 112 is formed in the mold 3 after finishing the freeze-drying process of the second supernatant. Subsequently, a first supernatant obtained from the step S5 is filled into the same mold 3, and a silk fibroin layer 111 is formed on the calcium-degradation silk fibroin layer 112 accommodated in the mold 3 after completing the freeze-drying process of the first supernatant.

Of course, manufacturing process described by FIG. 4A does not used for limiting the formation way of the silk fibroin layer 111 and the calcium-degradation silk fibroin layer 112. According to FIG. 4B, the second supernatant obtained from the step S7 and the first supernatant obtained from the step S5 can be filled into a first mold 3A and a second mold 3B, respectively. Subsequently, after applying a freeze-drying process to both the first supernatant and the second supernatant, a silk fibroin layer 111 and a calcium-degradation silk fibroin layer 112 are formed in the first mold 3A and the second mold 3B, respectively. As a result, there is a double-layer dressing 1 containing silk fibroin produced by connecting the calcium-degradation silk fibroin layer 112 to the silk fibroin layer 111. It is worth further explaining that, the silk fibroin layer 111 and the calcium-degradation silk fibroin layer 112 respectively have a first mean pore area and a second mean pore area, and the first mean pore area being larger than the second mean pore area. Moreover, adding an ethanol solution into the mixture solution obtained from the step S6 is helpful for reducing the porosity of the calcium-degradation silk fibroin layer 112 obtained from the step S8.

### Second Embodiment

Please refer to FIG. 1 again. The present invention further discloses a second embodiment for the double-layer dressing 1 containing silk fibroin, comprising: a silk fibroin layer 111, a calcium-degradation silk fibroin layer 112 disposed on the silk fibroin layer 111, and a wound healing promoting agent, wherein the wound healing promoting agent is contained in the silk fibroin layer 111 and/or the calcium-degradation silk fibroin layer 112. Particularly speaking, chlorhexidine diacetate is one kind of antibacterial agent commonly contained in commercial antibacterial wound dressing. Therefore, the chlorhexidine diacetate can be used as the wound healing promoting agent so as to be contained in the silk fibroin layer 111 and/or the calcium-degradation silk fibroin layer 112. Moreover, the said wound healing promoting agent can also be selected from the group consisting of Ag ions, water extract of seasoned orange peels containing hesperidin, water extract of Gastrodia elata blume containing gastrodin, citrus polyphenols, composition comprising at least one vitamin and hyaluronic acid, extract of salmon, composition comprising α-helix peptide and short peptide, extract of Cassiopea andromeda, lycopene, extract of explant of Saussurea involucrate, 4,7-dimethoxy-5-methyl-1,3-benzodioxole extracted from Antrodia camphorate, composition comprising ethanol extract of Japanese honeysuckle and water extract of peppermint, fungal immunomodulatory protein LZ-8, extract of coral, hemocyanin, and composition comprising extract of Plectranthus amboinicus and extract of Asiatic centella.

Mechanical Testing

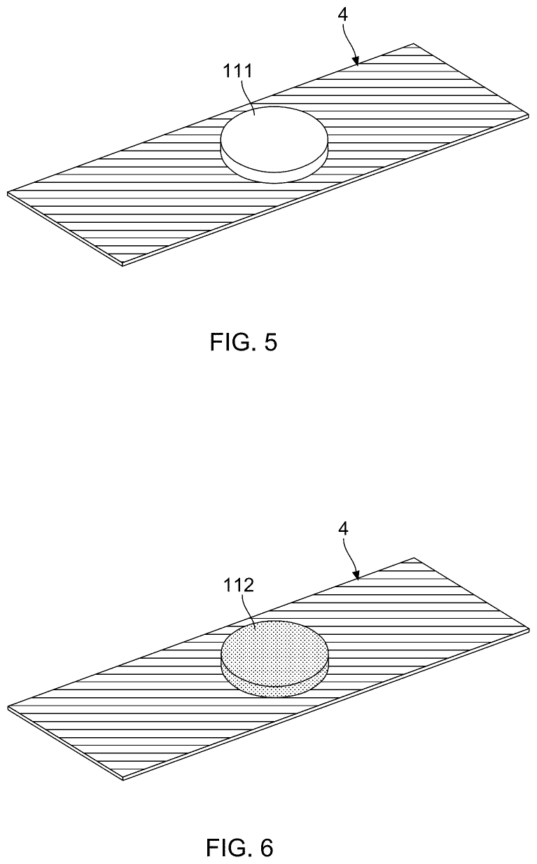

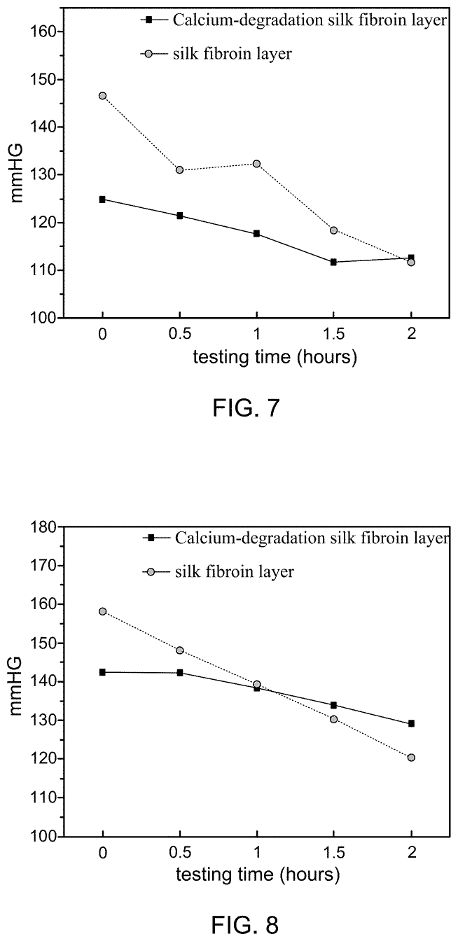

FIG. 5 shows a sample of the silk fibroin layer being subject to mechanical testing, and FIG. 6 shows a sample of the calcium-degradation silk fibroin layer being subject to mechanical testing. To execute the mechanical testing, both the silk fibroin layer 111 and the calcium-degradation silk fibroin layer 112 are disposed on one respective elastic bandage 4. Subsequently, the elastic bandage 4 is pulled to extent by 4 cm, and then is kept at such condition for 2 hours. FIG. 7 shows a plot graph of mmHg versus testing time. From the data of FIG. 7, it is found that, the (compressing) pressure provided by the silk fibroin layer 111 still exceeds 110 mmHg even if the elastic bandage 4 is pulled to extent by 4 cm for 2 hours. Moreover, data of FIG. 7 also exhibits that the (compressing) pressure provided by the calcium-degradation silk fibroin layer 112 still exceeds 110 mmHg in spite of the fact that the elastic bandage 4 is pulled to extent by 4 cm for 2 hours.

Furthermore, both the silk fibroin layer 111 and the calcium-degradation silk fibroin layer 112 are disposed on another respective elastic bandage 4, in order to execute another mechanical testing. Subsequently, the elastic bandage 4 is pulled to extent by 5 cm, and then is kept at such condition for 2 hours. FIG. 8 shows a plot graph of mmHg versus testing time. From the data of FIG. 8, it is found that, the (compressing) pressure provided by the silk fibroin layer 111 still exceeds 120 mmHg even if the elastic bandage 4 is pulled to extent by 5 cm for 2 hours. Moreover, data of FIG. 8 also exhibits that the (compressing) pressure provided by the calcium-degradation silk fibroin layer 112 still exceeds 110 mmHg in spite of the fact that the elastic bandage 4 is pulled to extent by 5 cm for 2 hours.

Animal Experiment

Inventors of the present invention have finished two animal experiments using rabbits as experiment animals. Please refer to following Table (1), there are three groups designed in a first experiment. Moreover, there are four executing steps for completing the first experiment, including:
(1) using corresponding test samples to cover the wound on respective rabbit' skin in each group for 300 seconds;
(2) removing the test sample from the rabbit' skin, and letting time passing 300 seconds;
(3) covering the wound on respective rabbit' skin in each group by new corresponding test samples, for 300 seconds; and
(4) removing the test sample from the rabbit' skin, and letting time passing 300 seconds.

TABLE 1

| Groups | Test samples |
|---|---|
| Blank | Silk fibroin layer 111 as shown in FIG. 1 |
| Experiment | Double-layer dressing 1 (as shown in FIG. 1) |
| | comprising one silk fibroin layer 111 and one |
| | calcium-degradation silk fibroin layer 112 |
| | disposed on the silk fibroin layer 111. |
| Control | Commercial wound dressing |

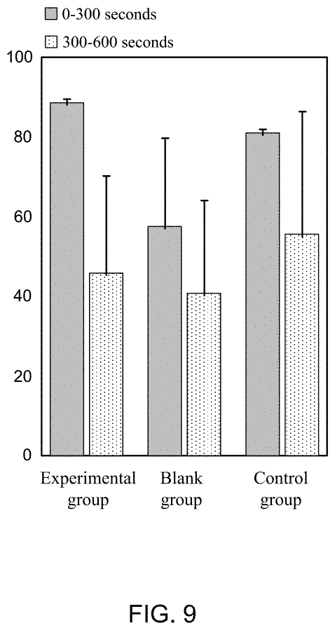

Experimental data of the first experiment have integrated and recorded in following Table (2) and Table (3), and FIG. 9 shows a statistical bar graph of various test samples for describing blood absorption ability of the test samples. From Table (2), it is understood that, in the case of directly covering the wound, both the double-layer dressing 1 and the silk fibroin layer 111 exhibit outstanding hemostasis efficiency better than that of the commercial wound dressing. Moreover, data of Table (3) show that the double-layer dressing 1 has a good anti-adhesion performance superior than the that of the silk fibroin layer 111.

TABLE 2

| Group | Time for achieving hemostasis (s) |
|---|---|
| Blank | >600 |
| Experiment | 102.5 ± 5.57 |
| Control | 369 ± 79.2 |

TABLE 3

| | | |
|---|---|---|
| N = 4 | Adhesion index | Average |

| | | | | | |
|---|---|---|---|---|---|
| Blank group | 0 | 1 | 0 | 1 | 0.50 ± 0.58 |
| Experiment group | 0 | 0 | 0 | 1 | 0.25 ± 0.50 |
| Control group | 0 | 1 | 0 | 0 | 0.25 ± 0.50 |

Please refer to following Table (4), there are three groups designed in a second experiment. It is worth further explaining that the rabbits have a normal systolic blood pressure (SBP) range of 90-120 mmHg. In the second experiment, the elastic bandage of corresponding test sample is pulled to extent by 5 cm, and then used to cover the wound on respective rabbit's skin.

TABLE 4

| Groups | Test samples |
|---|---|
| Blank | Silk fibroin layer 111 as shown in FIG. 1 |
| Experiment | Double-layer dressing 1 (as shown in FIG. 1) |
| | comprising one silk fibroin layer 111 and one |
| | calcium-degradation silk fibroin layer 112 |
| | disposed on the silk fibroin layer 111. |
| Control | Commercial wound dressing |

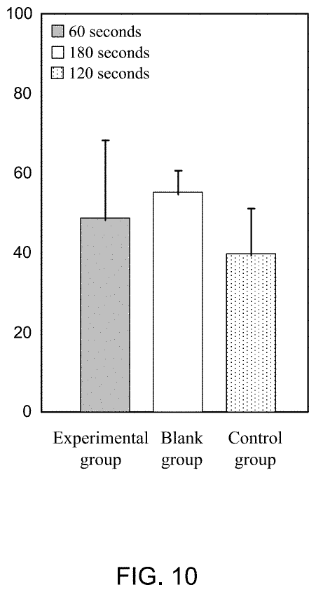

Experimental data of the second experiment have integrated and recorded in following Table (5) and Table (6), and FIG. 10 shows a statistical bar graph of various test samples for describing blood absorption ability of the test samples. From Table (5), it is understood that, in the case of directly covering the wound, both the double-layer dressing 1 and the silk fibroin layer 111 exhibit outstanding hemostasis efficiency better than that of the commercial wound dressing. Moreover, data of Table (6) show that the double-layer dressing 1 has a good anti-adhesion performance superior than the that of the silk fibroin layer 111.

TABLE 5

| Group | Time for achieving hemostasis (s) |
|---|---|
| Blank | 180 |
| Experiment | 60 |
| Control | 120 |

TABLE 6

| | | |
|---|---|---|
| N = 4 | Adhesion index | Average |

| | | | | | |
|---|---|---|---|---|---|
| Blank group | 0 | 0 | 1 | 0 | 0.25 ± 0.50 |
| Experiment group | 0 | 0 | 0 | 1 | 0.25 ± 0.50 |
| Control group | 0 | 0 | 1 | 0 | 0.25 ± 0.50 |

Therefore, through above descriptions, the double-layer dressing containing silk fibroin provided by the present invention has been introduced completely and clearly; in summary, the present invention includes the advantages of:

(1) The present invention provides a double-layer dressing containing silk fibroin and a method for making the same. The double-layer dressing mainly comprises a silk fibroin layer and a calcium-degradation silk fibroin layer connected to the silk fibroin layer. It is worth emphasizing that, results of animal experiment have proved that this novel double-layer dressing is an outstanding hemostatic wound dressing. Moreover, additional adhesion, resulted from the solidification of tissue fluid, can be effectively prevented from forming between skin wound and wound dressing under the use of this double-layer dressing.

The above description is made on embodiments of the present invention. However, the embodiments are not intended to limit scope of the present invention, and all equivalent implementations or alterations within the spirit of the present invention still fall within the scope of the present invention.

What is claimed is:

1. A double-layer dressing containing silk fibroin, comprising:
a silk fibroin layer; and
a calcium-degradation silk fibroin layer, being disposed on the silk fibroin layer.

2. The double-layer dressing containing silk fibroin of claim 1, further comprising:
a substrate, being used for supporting the silk fibroin layer by the surface thereof.

3. The double-layer dressing containing silk fibroin of claim 1, wherein the silk fibroin layer and the calcium-degradation silk fibroin layer respectively have a first mean pore area and a second mean pore area, and the first mean pore area being larger than the second mean pore area.

4. The double-layer dressing containing silk fibroin of claim 1, wherein a wound healing promoting agent is contained in the silk fibroin layer and/or the calcium-degradation silk fibroin layer.

5. A method for making double-layer dressing containing silk fibroin, comprising following steps:
(1) preparing a raw material of silkworm cocoon, and then producing a silk fibroin product by applying a degumming process to the raw material of silkworm cocoon;
(2) dissolving the silk fibroin product in a salt solution, so as to obtained a silk fibroin solution;
(3) applying a dialysis process to the silk fibroin solution, thereby obtaining a dialysate of silk fibroin;
(4) separating the dialysate of silk fibroin into a first dialysate and a second dialysate;
(5) producing a first supernatant by applying a first centrifuging process to the first dialysate;
(6) applying a degradation process to the second dialysate so as to obtain a protein-degraded solution, and subsequently producing a mixture solution by mixing the protein-degraded solution with a solution of bivalent metal salt;
(7) producing a second supernatant by applying a second centrifuging process to the mixture solution;
(8) applying a freeze-drying process to both the first supernatant and the second supernatant thereby respectively obtaining a silk fibroin layer and a calcium-degradation silk fibroin layer, and then producing a double-layer dressing containing silk fibroin by disposing the calcium-degradation silk fibroin layer on the silk fibroin layer.

6. The method of claim 5, wherein the silk fibroin layer obtained from the step (8) is further immersed in a wound healing promoting agent, so as to make the wound healing promoting agent be contained in the silk fibroin layer.

7. The method of claim 5, wherein the calcium-degradation silk fibroin layer obtained from the step (8) is further immersed in a wound healing promoting agent, so as to make the wound healing promoting agent be contained in the calcium-degradation silk fibroin layer.

8. The method of claim 5, wherein further adding an ethanol solution into the mixture solution obtained from the step (6) is helpful for reducing the porosity of the calcium-degradation silk fibroin layer obtained from the step (8).

9. The method of claim 5, wherein the solution of bivalent metal salt comprises a bivalent metal salt and a solution, and having a solution concentration of at least 0.2 mg/mL.

10. The method of claim 5, wherein the degradation process is achieved by adding a protease solution comprising a buffer solution and a protease into the second dialysate.

11. The method of claim 9, wherein the solution is water, and the bivalent metal salt being selected from the group consisting of zinc chloride, calcium chloride and magnesium chloride.

12. The method of claim 10, wherein the protease is selected from the group consisting of protease produced by Bacillus amyloliquefaciens, protease produced by Streptomyces griseus, α-chymotrypsin, chymotrypsin, and carboxylase.

13. The method of claim 10, wherein the protease has a molecular weight in a range from 25000 daltons to 35000 daltons.

14. The method of claim 10, wherein the buffer solution is selected from the group consisting of phosphate buffered saline (PBS) and deionized water.
